(19) United States
(12) Patent Application Publication
(10) Pub. No.: US 2005/0095690 A1
(43) Pub. Date: May 5, 2005

(54) Entrapment of biomolecules and inorganic nanoparticles by biosilicification

(76) Inventors: Rajesh R. Naik, Centerville, OH (US); Morley O. Stone, Bellbrook, OH (US); Jim C. Spain, Panama City, FL (US); Heather R. Luckarift, Panama City, FL (US)

Correspondence Address: TRASK BRITT, P.O. BOX 2550, SALT LAKE CITY, UT, 84110, US

(21) Appl. No.: 10/803,633

(22) Filed: Mar. 18, 2004

Related U.S. Application Data
(60) Provisional application No. 60/517,227, filed on Oct. 31, 2003.

(51) Int. Cl. C12P 21/04; C12P 3/00

(52) U.S. Cl. 435/168

(57) ABSTRACT

A method of immobilizing at least one molecule in a silica matrix to form a biosilicification product. The at least one molecule may be immobilized in the silica matrix at substantially the same time as the silica matrix is formed. The method comprises combining at least one silaffin polypeptide, at least one molecule, and at least one hydroxylated water-soluble derivative to form the biosilicification product. The silaffin polypeptide may be Sil1 protein from C. fusiformis, a fragment of the Sil1 protein, poly-L-lysine, or a synthetic polypeptide having affinity for silica. The at least one molecule may be an enzyme, a protein, a polypeptide, an antibody, an antigen, poly(nucleic) acids, microbial cells, plant cells, or animal cells. The hydroxylated water-soluble derivative may be silicic acid.

### GOVERNMENT RIGHTS

This invention was made with Government support under Contract No. F33615-01-C-5214 awarded by the Department of the Air Force. The Government has certain rights in this invention.

### CROSS-REFERENCE TO RELATED APPLICATION

This application claims the benefit of U.S. Provisional Patent Application Ser. No. 60/517,227, filed Oct. 31, 2003, for ENTRAPMENT OF BIOMOLECULES AND INORGANIC NANOPARTICLES BY BIOSILICIFICATION.

### FIELD OF THE INVENTION

The present invention relates to a biological approach for encapsulating, immobilizing, or entrapping a biological or nonbiological material in a silica matrix. More specifically, the present invention relates to a method of encapsulating the biological or nonbiological material in the silica matrix as the silica matrix is formed.

### BACKGROUND OF THE INVENTION

Biomolecules, such as enzymes, proteins, and cells, have been immobilized in matrices and used in catalysis and sensors. The biomolecules are typically used to catalyze a desired reaction or to sense or detect a desired analyte. To preserve biological activity of the biomolecule, the matrix in which the biomolecule is immobilized is carefully selected. Silicates, such as sol-gel composites or mesoporous silica, have been extensively used to immobilize a wide variety of biomolecules. One of the most widely used immobilization techniques is sol-gel silica encapsulation in which an alkoxysilane precursor is hydrolyzed by water, acid, or base catalysis to form a sol of soluble hydroxy derivatives. A buffered solution including the biomolecule is subsequently added to the hydroxy derivatives. Then, polymerization and gelation is initiated to form a hydrated oxide hydrogel, which is slowly aged at low temperature over a period of several weeks.

One limitation of sol-gel silica encapsulation is that the hydrolysis of the alkoxysilane precursor produces alcohol byproducts. The presence of these alcohol byproducts in the sol-gel silica encapsulation process decreases the biological activity of the immobilized biomolecule. In addition, extreme processing conditions, such as extreme pH, high temperatures, and high pressures, are necessary to synthesize the sol-gel, which decreases the activity of the biomolecule. The resulting sol-gel also includes water and alcohols, which must be removed before the encapsulated material is used. However, as the water or alcohol is removed, pores in the encapsulated material collapse, decreasing its stability. The pore collapse also leads to decreased activity because the biomolecule is no longer readily accessible to the surrounding environment. Sol-gel silica encapsulation is also limited by an amount of the biomolecule that is loaded onto the matrix. Sol-gel processes are disclosed in U.S. Pat. No. 3,821,083 to Van Leemputten et al., U.S. Pat. No. 5,951,962 to Müller et al., U.S. Pat. No. 6,395,299 to Babich et al., U.S. Pat. No. 6,495,352 to Brinker et al., and U.S. Pat. No. 5,874,109 to Ducheyne et al.

In contrast, mesoporous silica appears to be an improved matrix in which to immobilize the biomolecules compared to the sol-gel composites. The biomolecule is immobilized in the pores of the mesoporous silica by incorporating functional groups to which the biomolecule has high affinity in the pores. However, using the functionalized mesoporous silica is not optimal because the affinity of the biomolecule for the functional group is highly specific. Therefore, the functional groups on the mesoporous silica must be specifically optimized for each type of biomolecule that is to be immobilized. In addition, the biomolecule is only added after the mesoporous silica has been formed, which adds additional steps and complexity to the process. Mesoporous silica processes are disclosed in WO 02/068454 to Ackerman et al., EP 1251184 to Kajino et al., U.S. Patent Application Publication 2002/0015985 to Takahashi et al.

Diatoms, which are unicellular, eukaryotic algae, produce silica-based materials, such as silica-based cell walls. These silica structures are produced under mild physiological conditions and, therefore, the diatoms' mechanisms for producing these structures have been the subject of intense research. Silica-based materials, such as nanostructured silica, have been produced in vitro at neutral pH, ambient temperature, and ambient pressure by adding silaffin polypeptides to a solution of silicic acid, as disclosed in Kröger et al., “Polycationic Peptides from Diatom Biosilica that Direct Silica Nanosphere Formation,” Science, 286:1129-1132 (1999) (“Kröger 1”), Kröger et al., “Self-Assembly of Highly Phosphorylated Silaffins and Their Function in Biosilica Morphogenesis,” Science, 298:584-586 (2002) (“Kröger 2”), and Naik et al., “Controlled formation of biosilica structures in vitro,” Chem. Commun. 2:238-239 (2003) (“Naik 1”), the disclosure of each of which is incorporated by reference herein. As used herein, the term “silaffin polypeptide” refers to a polypeptide having affinity for silica and that is capable of precipitating silica. The silaffin polypeptides are typically cationic polypeptides isolated from the diatom Cylindrotheca fusiformis or synthetic peptides that are capable of precipitating silica. The silaffin polypeptide catalyzes the formation of the nanostructured silica.

### BRIEF SUMMARY OF THE INVENTION

The present invention relates to a method of immobilizing at least one molecule in a silica matrix. As used herein, the terms “a molecule” or “the molecule” refer to at least one molecule and, therefore, include one molecule or a plurality of molecules. The molecule and the silica matrix comprise a biosilicification product. The molecule may be immobilized in the silica matrix at substantially the same time as the silica matrix is formed, which enables the biosilicification product to be produced in a one-pot process. The biosilicification product may also be produced under mild conditions that do not decrease activity of the molecule. The method comprises combining at least one silaffin polypeptide, a molecule, and at least one hydroxylated water-soluble derivative. The silaffin polypeptide may be a polypeptide selected from the group consisting of Sil1 protein from C. fusiformis, a fragment of the Sil1 protein, and poly-L-lysine. The silaffin polypeptide may have an amino acid sequence selected from the group consisting of SEQ ID NO:1, SEQ ID NO:2, SEQ ID NO:3, SEQ ID NO:4, SEQ ID NO:5, SEQ ID NO:6, and SEQ ID NO:7. The hydroxylated water-soluble derivative may be silicic acid.

The molecule may be an organic molecule, such as an enzyme, a protein, a polypeptide, an antibody, an antigen, poly(nucleic) acids, microbial cells, plant cells, or animal cells. Examples of enzymes that may be used include, but are not limited to, hydrolases, catalases, or esterases, such as butyrylcholinesterase, horseradish peroxidase (“HRP”), or catalase. Alternatively, the molecule may be an inorganic molecule, such as a magnetic material or a semiconductor material. Examples of semiconductor materials include, but are not limited to, cadmium selenide, cadmium sulfide, cadmium telluride, zinc selenide, zinc sulfide, or zinc telluride.

The biosilicification product may be formed at a pH ranging from approximately 5 to approximately 8, such as at a pH of approximately 7. The biosilicification product may be formed at a temperature ranging from approximately 10° C. to approximately 25° C. and at ambient pressure.

### BRIEF DESCRIPTION OF THE SEVERAL VIEWS OF THE DRAWINGS

While the specification concludes with claims particularly pointing out and distinctly claiming that which is regarded as the present invention, the advantages of this invention can be more readily ascertained from the following description of the invention when read in conjunction with the accompanying drawings in which:

FIGS. 1A and 1B are each a scanning electron microscopy (“SEM”) micrograph of a butyrylcholinesterase immobilized product;

FIG. 2 is an SEM micrograph of a catalase immobilized product;

FIG. 3 shows relative activity of the immobilized catalase product and the immobilized HRP product compared to free soluble catalase and free soluble HRP;

FIG. 4 shows that the immobilized catalase product is resistant to protease degradation and heat;

FIG. 5 demonstrates the presence of catalase within the silica matrix and the protection of immobilized catalase from protease degradation; in contrast, free catalase is degraded in the presence of a protease;

FIG. 6 shows stability of butyrylcholinesterase activity at 25° C. in the immobilized butyrylcholinesterase product (●) compared to that of the free butyrylcholinesterase ((free enzyme (▪) and the free enzyme with antibiotic solution (♦)); and

FIG. 7 shows the stability of the immobilized butyrylcholinesterase product in a flow-through reaction system. Activity of the immobilized butyrylcholinesterase product was compared in a packed-bed system (♦) and a fluidized-bed system (▴). The concentration of the hydrolysis product (absorbance at 630 nm) was measured in the eluate. The feed solution of indophenyl acetate (2×10⁻⁴M) was supplied at a flow-rate of 2 ml/min. At the point indicated by an arrow (↓), the immobilized butyrylcholinesterase product was removed from the column, washed by centrifugation, and returned to the column.

### DETAILED DESCRIPTION OF THE INVENTION

A method of immobilizing at least one molecule in a silica matrix to form a biosilicification product is disclosed. As previously discussed, the terms “a molecule” or “the molecule” refer to at least one molecule, such as one molecule or a plurality of molecules. In other words, the biosilicification product may include at least one molecule immobilized in the silica matrix. The silica matrix may be formed by a biomimetic process that utilizes mild reaction conditions. As such, the biosilicification product may be formed without decreasing activity of the molecule. The molecule may be incorporated into the silica matrix as the silica matrix is formed, enabling production of the biosilicification product in a one-pot process. The biosilicification product may be used as a catalyst or as a sensor. For instance, the biosilicification product may be used to catalyze a desired reaction of a substrate to a product. Alternatively, the biosilicification product may be used to sense or detect an analyte.

The silica matrix may be formed in vitro under mild reaction conditions. Since the silica matrix is formed by a biomimetic process, it is also referred to herein as biosilica. The silica matrix may includes particles or nanospheres of biosilica that have a diameter ranging from approximately 400 nm to approximately 600 nm. For instance, the biosilica may have an average diameter of 500 nm. The silica matrix may include pores having a pore size of less than approximately 100 nm. For instance, the silica matrix may have pores ranging from approximately 10 nm to approximately 100 nm, such as from approximately 10 nm to approximately 15 nm. The silica matrix may be a network of fused silica particles. The matrix in which the molecule is immobilized may also be a titanium-containing material, such as titanium dioxide.

The silica matrix of the biosilicification product may be formed by a biosilicification reaction in which a silaffin polypeptide condenses a hydroxylated water-soluble derivative. In other words, the silaffin polypeptide catalyses a condensation reaction of the hydroxylated water-soluble derivative. The silica matrix may be formed by combining at least one hydroxylated water-soluble derivative and at least one silaffin polypeptide at a pH ranging from approximately 5 to approximately 8. For instance, the pH may be approximately 7. The biosilicification reaction may be performed under mild reaction conditions, such as at a temperature ranging from approximately 10° C. to approximately 25° C. and at ambient pressure. For instance, the biosilicification reaction may be performed at room temperature (approximately 25° C.) and at ambient pressure.

The hydroxylated water-soluble derivative may be a silicon-containing derivative, such as silicic acid. As previously described in Kröger 1, Kröger 2, and Naik, silaffin polypeptides have been isolated from diatoms. The silaffin polypeptide may have affinity for silica and may precipitate silica. In other words, the silaffin polypeptide may catalyze a condensation reaction of the hydroxylated water-soluble derivative to form the silica. The silaffin polypeptide may be Sil1 protein from C. fusiformis, a fragment of the Sil1 protein, poly-L-lysine, a synthetic polypeptide capable of precipitating silica, or mixtures thereof. The Sil1 protein is a 265 amino acid residue polypeptide having seven highly homologous repeating units known as R1 to R7. The silaffin polypeptides from C. fusiformis include lysine residues that are linked by their ε-amino groups to long-chain polyamines. For instance, the silaffin polypeptide may be a 19 amino acid, R5 unit peptide of the Sil1 protein, which has the amino acid sequence of H₂N-SSKKSGSYSGSKGSKRRIL-OH (SEQ ID: NO. 1) and is referred to herein as the R5 peptide.

The silaffin polypeptide may also be a 12 amino acid peptide having affinity for silica, as disclosed in Naik et al., “Silica-Precipitating Peptides Isolated from a Combinatorial Phage Display Peptide Library,” J. Nanosci. Nanotech., 2:95-100 (2002) (“Naik 2”), which is incorporated by reference herein. The silaffin polypeptide may have one of the following amino acid sequences:

| | | |
|---|---|---|
| Si3-3 | H₂N-APPGHHHWHIHH-OH | (SEQ ID: NO.2) |
| Si3-8 | H₂N-KPSHHHHHTGAN-OH | (SEQ ID: NO.3) |
| S14-1 | H₂N-MSPHPHPRHHHT-OH | (SEQ ID: NO.4) |
| Si4-3 | H₂N-MSPHHMHHSHGH-OH | (SEQ ID: NO.5) |
| Si4-7 | H₂N-LPHHHHLHTKLP-OH | (SEQ ID: NO.6) |
| Si4-8 | H₂N-APHHHHPHHLSR-OH | (SEQ ID: NO.7) |

When the silaffin polypeptide and the hydroxylated water-soluble derivative are combined, the silica matrix may be formed by the biosilicification reaction, in which the silaffin polypeptide causes the hydroxylated water-soluble derivative to precipitate. Specifically, the silaffin polypeptide may condense the hydroxylated water-soluble derivative, causing biosilica particles to precipitate, thus forming the silica matrix. The silaffin polypeptide may precipitate the silica matrix within seconds after the silaffin polypeptide is added to the hydroxylated water-soluble derivative.

In one embodiment, the hydroxylated water-soluble derivative is silicic acid. The silicic acid may be formed by hydrolyzing tetramethylorthosilicate (“TMOS”), tetraethyl-orthosilicate (“TEOS”), or sodium silicate with an acid, such as hydrochloric acid. In one embodiment, the silaffin polypeptide is the R5 peptide. The R5 peptide may precipitate the silica matrix within seconds when added to the solution of silicic acid.

The molecule immobilized in the silica matrix may be a biological material or a nonbiological material, such as an organic molecule, an inorganic molecule, or mixtures thereof. Any molecule having a sufficiently small size to be immobilized in the pores of the silica matrix may be used. For instance, the organic molecule may be a hydrophilic compound that is soluble in the hydroxylated water-soluble derivative and the silaffin polypeptide. Alternatively, the organic molecule may be a biomolecule, such as an enzyme, a protein, a polypeptide, an antibody, an antigen, poly(nucleic) acids, live microbial cells, live plant cells, live animal cells, or mixtures thereof. For sake of example only, the biomolecule may be a catalase, an esterase, or a hydrolase enzyme. Specific examples of enzymes that may be used include, but are not limited to, butyrylcholinesterase, HRP, or catalase. The molecule may also be an inorganic molecule, such as an inorganic magnetic material, an inorganic semiconductor material, or mixtures thereof. The inorganic molecule may have a size of less than approximately 200 nm, such as a size ranging from approximately 10 nm to approximately 50 nm. For sake of example only, the inorganic molecule may be cobalt platinum, iron oxide, titanium dioxide, cadmium selenide, cadmium sulfide, cadmium telluride, zinc selenide, zinc sulfide, zinc telluride, or mixtures thereof. While the Examples herein describe the molecule as an enzyme, it is understood that the molecule may be another type of biomolecule, an organic molecule, or an inorganic molecule.

To form the biosilicification product, the molecule may be solubilized in an aqueous solution having a pH suitable to preserve the activity of the molecule. For instance, the molecule may be solubilized in a buffered, aqueous solution. The molecule may then be combined with a solution of the silaffin polypeptide. The solution of the molecule and the silaffin polypeptide may then be added to a solution of the hydroxylated water-soluble derivative to form a biosilicification reaction mixture. Alternatively, the molecule may be combined with the hydroxylated water-soluble derivative and then added to the silaffin polypeptide. The reaction of the molecule, the silaffin polypeptide, and the hydroxylated water-soluble derivative is referred to herein as the immobilization reaction. The term “immobilization reaction” is used to distinguish this reaction from the biosilicification reaction, in which only the silaffin polypeptide and the hydroxylated water-soluble derivative are reacted. The immobilization reaction may be performed at a pH ranging from approximately 5 to approximately 8. For instance, the pH may be approximately 7. The immobilization reaction may be performed at a temperature ranging from approximately 10° C. to approximately 25° C. and at ambient pressure. For instance, the immobilization reaction may be performed at room temperature (approximately 25° C.) and at ambient pressure.

Since the silaffin polypeptide precipitates the hydroxylated water-soluble derivative quickly, the silica matrix may be formed and the molecule immobilized within the silica matrix substantially simultaneously. Furthermore, since the immobilization reaction is performed under mild reaction conditions, the silica matrix may be synthesized in the presence of the molecule to be immobilized without affecting the activity of the molecule. The precipitated biosilica may then be separated from solution, producing the biosilicification product having the molecule immobilized in the silica matrix. The immobilized molecule may be entrapped in the pores of the silica matrix, which provides a stable environment for the immobilized molecule. As such, the activity of the immobilized molecule may be preserved even when the biosilicification product is exposed to room temperature for extended time periods or elevated temperatures for shorter time periods.

The molecule may be present in the biosilicification product in an amount sufficient to perform the desired catalysis or sensor function. If the molecule is an enzyme, the enzyme loading may range from approximately 0.7% (w/w) to approximately 21% (w/w), depending on the enzyme. For instance, if the enzyme is butyrylcholinesterase, the enzyme loading may be up to approximately 220 mg enzyme/g silica matrix (20% w/w). In contrast, the enzyme loading in conventional sol-gel protocols is usually limited to 0.1-5% w/w, due primarily to the hydrophobicity of alkyl silicates, the denaturing effect of alcohol byproducts, and protein aggregation at elevated concentrations.

The biosilicification product produced by the immobilization reaction may have an increased stability compared to the stability of an immobilized molecule encapsulated by a conventional sol-gel process. The stability is improved because the biosilicification product is formed from a solid material. As such, the pores are stable and do not collapse when the biosilicification product is dried. Since the pores retain their porosity, the biosilicification product retains its stability after the biosilicification product is dried. In addition, the porous nature of the silica matrix maintains the activity of the molecule by allowing access and diffusion into the biosilicification product. Therefore, the biosilicification product formed by the biosilicification reaction provides a stable matrix into which the molecule is immobilized.

Since the biosilicification product is stable, the biosilicification product may be used in a variety of products that would not be feasible with a soluble enzyme system. For instance, the biosilicification product may be used in a continuous flow system or a flow-through system, such as a fluidized-bed system or a packed-bed system. The flow-through system may be a mini-reactor system in which the immobilized molecule is continually reused over an extended period of time. In batch systems, the immobilized molecule may be recovered from the reaction media and reused, leading to significant cost reduction.

The physical morphology of the biosilicification product may be manipulated by varying a reaction environment under which the biosilicification reaction is performed. For instance, if nitrogen gas is passed over the biosilicification reaction mixture during the immobilization reaction, the biosilicification product may adopt an archlike structure, as described in Naik 1. Alternatively, if shear is applied to the biosilicification reaction mixture during the immobilization reaction, the biosilicification product may form elongated fibers. For sake of example only, the shear may be applied by cycling the biosilicification reaction mixture back and forth inside a reaction vessel during the immobilization reaction. By adjusting the reaction environment, desired shapes or morphologies of the biosilicification product may be produced. For instance, frits or filters including the biosilicification product may be formed by adjusting the reaction environment. These frits or filters may be incorporated into an immobilized enzyme system in a desired device for detection or catalysis.

The ability to immobilize a variety of molecules on the silica matrix may provide opportunities to combine disparate biological functionalities into new architectures. The biomimetic, biosilicification process described herein provides a simple route to a structurally defined, nanoporous support matrix, which may provide the basis for the development of biosensors, decontamination systems, and immobilized enzyme reactors. The immobilized molecule may be significantly more stable than the free enzyme and the resulting biosilicification product may have improved mechanical properties that facilitate application in a flow-through reactor. Therefore, the use of biosilica to immobilize the molecule combines the excellent support properties of the silica matrix with a benign method for immobilization that permits retention of the molecule's activity.

The following examples serve to explain embodiments of the present invention in more detail. These examples are not to be construed as being exhaustive or exclusive as to the scope of this invention.

### EXAMPLES

### Example 1

### Enzyme and Reagents

Butyrylcholinesterase (EC 3.1.1.8) was obtained from Sigma-Aldrich Co. (St. Louis, Mo.) as a highly purified lyophilized powder from equine serum containing approximately 50% protein and activity of 1200 units per mg protein. Enzyme stock solutions of the butyrylcholinesterase were prepared by dissolving the lyophilized enzyme in a cholinesterase specific buffer (0.1 N NaOH, 0.1 M KH₂PO₄, pH 8). The stock solutions were divided into aliquots of 50 U/ml based on the nominal units of activity as designated by the manufacturer. Catalase and HRP were also obtained from Sigma-Aldrich Co. Stock solutions of each of the catalase and HRP were prepared by dissolving the lyophilized enzyme in sodium phosphate buffer (pH 7.5) at a concentration of 10 mg/ml.

The R5 peptide was obtained from New England Peptide, Inc. (Gardner, MA) and a 100 mg/ml stock solution of the R5 peptide was prepared in deionized water. All other chemicals were of analytical grade and were obtained from Sigma-Aldrich Co. Silicic acid was prepared by hydrolyzing TMOS in 1 mM hydrochloric acid to give a final concentration of 1M.

### Example 2

### Preparation of Biosilicification Products

Biosilicification products were synthesized using the R5 peptide stock and silicic acid in the presence of the enzyme (butyrylcholinesterase, HRP, or catalase) to be immobilized. The silica matrix was prepared and the enzyme immobilized in a one-pot procedure.

To immobilize butyrylcholinesterase in the silica matrix, a biosilicification reaction mixture that included 80 μl of butyrylcholinesterase stock solution (3.5 μg protein), 10 μl of silicic acid (hydrolyzed TMOS) at a final concentration of 1.0 mg/ml, and 10 μl of the R5 peptide stock was prepared. The R5 peptide stock was added to a mixture of the butyrylcholinesterase stock solution and the silicic acid. The biosilicification reaction mixture was agitated for 5 minutes at room temperature (approximately 25° C.). The R5 peptide condensed the silicic acid, catalyzing the precipitation of silica within seconds. The precipitated silica particles were removed by centrifugation for 10 seconds (14,000×g) and then washed twice with deionized water. The resulting biosilicification product was a network of fused spherical silica particles with an average diameter of 500 nm. Butyrylcholinesterase was immobilized in the pores of the silica matrix. For sake of convenience and not by way of limitation, this biosilicification product is referred to herein as an immobilized butyrylcholinesterase product.

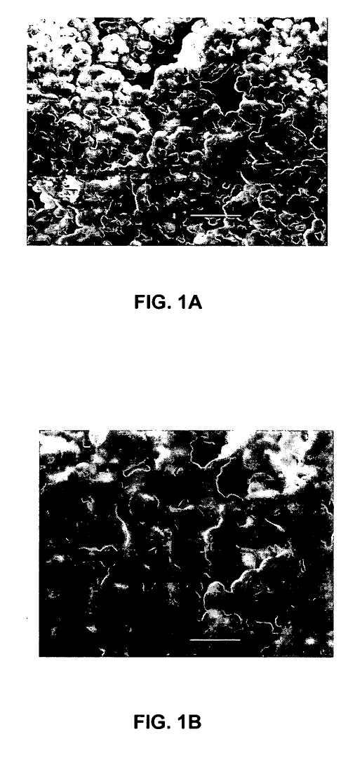

The immobilized butyrylcholinesterase product was analyzed by SEM to determine its morphology and size distribution. For SEM analysis, the immobilized butyrylcholinesterase product was washed with distilled water and mounted onto a carbon tape secured to an aluminum stub and observed under a Phillips XL30 FEG environmental scanning microscope. As shown in FIGS. 1A and 1B, the immobilized butyrylcholinesterase product was a matrix of fused silica nanospheres having an average diameter of 500 nm, which was in agreement with previous observations. In FIG. 1A, the scale bar is 2 μm; in FIG. 1B, the scale bar is 1 μm.

To immobilize HRP in the silica matrix, 10 mg/ml HRP was mixed with 10 μl of the R5 peptide stock and added to silicic acid (0.1M TMOS) in a sodium phosphate buffer having a pH of 7.5. The biosilicification reaction mixture was then incubated for 10 minutes at room temperature, allowing the silica to precipitate. The silica precipitate was collected and washed in a buffer containing 0.2% Tween-20. The washed silica precipitate was stored in a sodium phosphate buffer having a pH of 7.5. The resulting biosilicification product was a network of fused spherical silica particles having an average diameter of 500 nm. HRP was immobilized in the pores of the silica matrix. For sake of convenience, this biosilicification product is referred to herein as an immobilized HRP product.

To immobilize catalase in the silica matrix, 10 mg/ml catalase was mixed with 10 μl of the R5 peptide stock and added to silicic acid (0.1M TMOS) in a sodium phosphate buffer having a pH of 7.5. The biosilicification reaction mixture was then incubated for 10 minutes at room temperature, allowing the silica to precipitate. The silica precipitate was collected and washed several times in a buffer containing 0.2% Tween-20. The washed silica precipitate was stored in a sodium phosphate buffer having a pH of 7.5. The resulting biosilicification product was a network of fused spherical silica particles having an average diameter of 500 nm. Catalase was immobilized in the pores of the silica matrix. For sake of convenience, this biosilicification product is referred to herein as an immobilized catalase product.

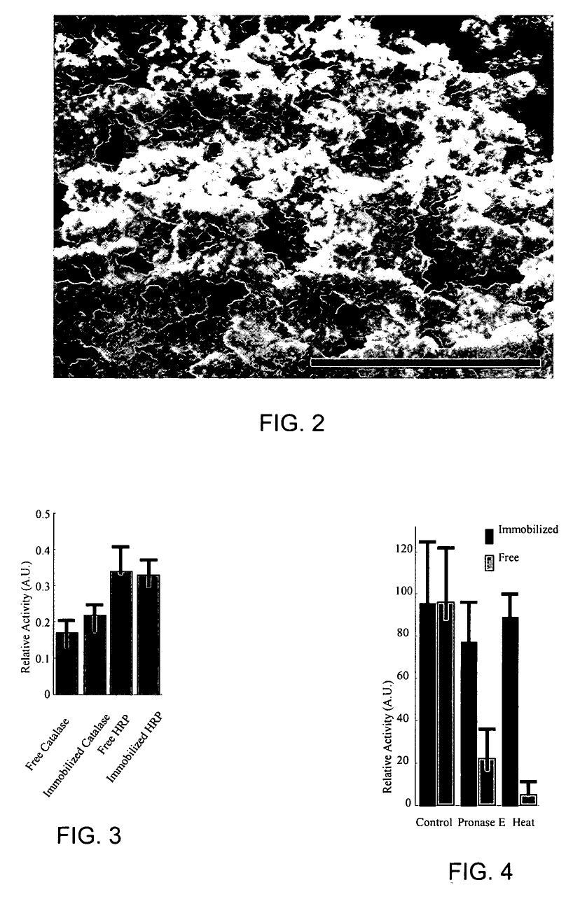

The immobilized catalase product was also analyzed by SEM. A network of large aggregates of fused silica particles was observed, as shown in FIG. 2 where the scale bar is 5 μm.

### Example 3

### Activity of the Immobilized Butyrylcholinesterase Product

The suitability of the silica matrix as an immobilization matrix for butyrylcholinesterase was determined with the immobilized butyrylcholinesterase product described in Example 2. To determine whether the butyrylcholinesterase retained its activity after the immobilization reaction, the activity of the butyrylcholinesterase entrapped in the silica matrix was compared with the activity of the free enzyme. Butyrylcholinesterase activity was measured spectrophotometrically at 630 nm using indophenyl acetate (2×10⁻⁴ M) as the substrate and a cholinesterase specific buffer (0.1 N NaOH, 0.1 M KH₂PO₄, pH 8). At pH 8.0, cholinesterases hydrolyze the yellow indophenyl acetate to a blue reaction product (4-(4-hydroxy-phenylimino)-cyclohexa-2,5-dienone). The absorptivity of the product was determined to be 8.1×10⁻³ M⁻¹ cm⁻¹ based on assumption of complete conversion by butyrylcholinesterase. Silica particles were removed by centrifugation (10 seconds at 14,000×g) before determination of absorbance. All assays were performed at room temperature unless otherwise stated. Protein concentration was determined by using a bicinchonic acid (“BCA”) protein assay kit (available from Pierce Biotechnology Inc.).

The immobilized butyrylcholinesterase product retained all of its activity compared to the activity of the free enzyme. Following immobilization of the enzyme, 90% (±7.2) of the initial free enzyme activity was detected in the silica matrix. Therefore, immobilization of the butyrylcholinesterase was 90% efficient. The remaining enzyme activity (5.4%±4.9) and protein was detected in the supernatant and water wash fractions, indicating negligible loss of enzyme activity following immobilization. The high efficiency of the immobilization technique is partly attributed to the mild conditions used to immobilize the enzyme. The mild encapsulation chemistry and high biocompatibility also eliminated any denaturing of the enzyme. In comparison, when butyrylcholinesterase was immobilized in a sol-gel process according to a previously reported method (described in Alstein et al., “Sol-gel entrapped cholinesterase: a mitrotitre plate method for monitoring anti-cholinesterase compounds,” J. Agric. Food. Chem. 46:3318-3324 (1998)), less than 10% of the initial enzyme activity remained (data not shown). The results obtained were in agreement with the previous study, which reported a 7-fold decrease in activity upon immobilization.

The efficiency of immobilization of the butyrylcholinesterase was investigated at a range of enzyme concentrations to determine the immobilization capacity of the silica matrix. The immobilization efficiency was reproducible at approximately 100% for enzyme concentrations up to 2 mg/ml. At an enzyme concentration of 4 mg/ml, the immobilization efficiency was reduced to 70%. The biosilicification reaction yielded approximately 1.2 mg of silica from a 100 μl reaction mixture. The calculated capacity for enzyme loading in the immobilized butyrylcholinesterase product was 220 mg enzyme/g silica (20% w/w).

To determine whether the butyrylcholinesterase was physically entrapped within the silica matrix during the immobilization reaction or simply adsorbed to the surface of the silica matrix, experiments were performed in which the silica matrix was prepared without adding butyrylcholinesterase. Then, the silica matrix was incubated with a solution of butyrylcholinesterase at the same enzyme concentration and reaction time as used in the immobilization reaction. The resulting precipitated biosilica particles were washed and all of the initial enzyme activity was detected in the wash fractions. These results indicate that when the immobilization reaction is used to form the butyrylcholinesterase immobilized product, the butyrylcholinesterase was physically entrapped and subsequently immobilized within the silica matrix. This conclusion was also confirmed by our observations that there was no significant leaching of the enzyme during continuous use.

### Example 4

### Activity of the Immobilized HRP and Catalase Products

The suitability of the silica matrix as an immobilization matrix was determined with the immobilized HRP and catalase products described in Example 2. To determine whether the HRP and the catalase retained activity in their respective biosilicification products, the activity of the immobilized HRP and catalase products was compared with the activity of the free enzymes. The activity of HRP was monitored using 2,2′-azino-bis(3-ethylbenzthiazoline-6-sulfonic acid) (“ABTS”) as the substrate. ABTS is a widely used substrate for HRP and produces a green-colored product that is read spectrophotometrically at 405 nm. As shown in FIG. 3, the immobilized HRP retained its activity and was comparable to that of free soluble HRP. The activity of the immobilized catalase was monitored by the decomposition of hydrogen peroxide into water and oxygen as measured by a decrease in absorbance at 240 nm. The activity of the immobilized catalase was also similar to that of the free soluble enzyme, as shown in FIG. 3.

These results indicated that HRP and catalase retained their activity during the biosilicification reaction. It was estimated that from approximately 15% to approximately 40% of the HRP or catalase added to the biosilicification reaction was immobilized within the silica matrix, while the remainder was recovered in the supernatant or from the buffer washes.

To determine whether the catalase and HRP were bound to the surface of the silica matrix or entrapped within the silica matrix, the catalase immobilized product and the HRP immobilized product were treated with a protease enzyme, pronase E. Free HRP was found to be protease resistant and was not further investigated. The activity of the treated catalase immobilized product was then determined. As shown in FIG. 4, treatment of the immobilized catalase product with pronase E did not greatly affect enzyme activity, indicating that the immobilized enzyme is not protease sensitive. In contrast, free catalase lost about 70% of its initial enzyme activity.

These results suggest that the catalase was most likely entrapped in the silica matrix during the immobilization reaction. While it is plausible that the immobilized catalase assumed a different conformation that was protease-resistant compared to the free enzyme, the ability of the immobilized catalase to bind to the substrate and process it in a similar way to the free soluble enzyme suggested that the catalase underwent normal conformational transitions during catalysis.

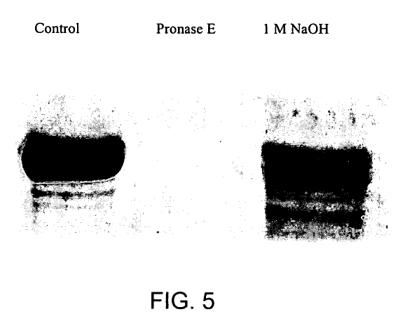

Further confirmation that the catalase was entrapped within the silica matrix was obtained using sodium dodecyl sulfate polyacrylamide gel electrophoresis (“SDS-PAGE”), as shown in FIG. 5. The immobilized catalase product having the immobilized catalase was dissolved in 1M sodium hydroxide (NaOH) for 20 minutes at 37° C. The dissolution of the silica matrix resulted in recovery of the catalase in the supernatant. The supernatant solution was then neutralized with 1M Tris-HCl (pH 7.5) and separated by SDS-PAGE. Catalase was released from the immobilized catalase product, as demonstrated by the presence of protein bands having a molecular weight of 60-65 kDa. The protein bands were also observed when the immobilized catalase product was treated with the protease, again confirming that the catalase is sequestered within the silica matrix. These results indicated that the catalase was immobilized in the silica matrix and only a fraction (<20%) was present on the surface of the immobilized catalase product.

Additional experiments were performed in which the silica matrix was prepared without adding the catalase. Then, the silica matrix was incubated with a solution of catalase at the same enzyme concentration and reaction time as used in the immobilization reaction. The resulting precipitated biosilica particles were washed several times in a sodium phosphate buffer including 0.2% Tween-20. After the washes, little or no enzyme activity was detected in the silica matrix while all of the initial enzyme activity was detected in the wash fractions, indicating that the washes removed most of the catalase bound to the surface of the silica matrix. These results further support that the immobilized catalase product formed by the immobilization reaction includes catalase that is physically entrapped and subsequently immobilized within the silica matrix.

### Example 5

### Stability of the Butyrylcholinesterase Immobilized Product

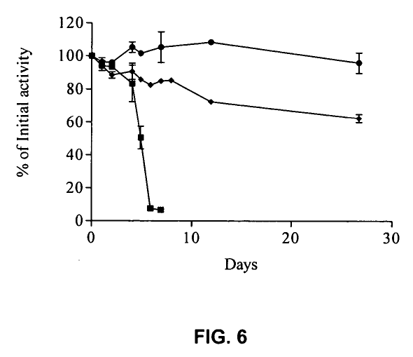

The stability of the butyrylcholinesterase during storage at 25° C. was evaluated over a 30-day period to determine whether the butyrylcholinesterase immobilized product is suitable for a wide range of applications. The stability during storage at 25° C. of the free enzyme in a buffer solution, of the free enzyme in the buffer solution including an antibiotic/antimycotic, and of the butyrylcholinesterase immobilized product in the buffer solution was determined. The antibiotic/antimycotic was purchased from Sigma-Aldrich Co. and included 10,000 units/ml penicillin G, 10 mg/ml streptomycin sulfate, and 25 μg/ml amphotericin B in 0.9% sodium chloride. For these long-term stability experiments, duplicates of free enzyme and the butyrylcholinesterase immobilized product were prepared with and without the addition of an antibiotic/antimycotic solution (Sigma) at a final concentration of 10 ml/L. As shown in FIG. 6, the immobilized enzyme retained 100% of the initial activity when stored in aqueous buffer for 30 days. In contrast, the free enzyme lost activity rapidly. The stability of the free enzyme was prolonged by the addition of the antibiotic/antimycotic solution, indicating that the loss of the enzyme activity was due to microbial degradation. The addition of the same antibiotic/antimycotic solution to the silica matrix did not change the stability. When the butyrylcholinesterase immobilized product was dried under vacuum before storage at 25° C., the activity was again retained for 30 days. The results indicate that immobilization of the butyrylcholinesterase in the silica matrix provided a stable environment for the enzyme and prevented the loss of activity that occurs when the enzyme is stored in solution at room temperature.

The thermostability of the free enzyme compared to the butyrylcholinesterase immobilized product was investigated to determine whether the silica matrix provided an environment that would protect the immobilized butyrylcholinesterase from thermal denaturation. The free enzyme in solution was denatured by incubation at 65° C. for 1 hour. These conditions resulted in an 85% decrease in activity compared to a control. In contrast, the butyrylcholinesterase immobilized product retained 100% of its activity when incubated under the same conditions. The ability to retain butyrylcholinesterase activity at high temperatures expands the range of conditions suitable for enzyme function and indicates a number of processing advantages for the immobilization reaction of the present invention, such as reduced risk of microbial contamination, lower viscosity, improved transfer rates, and improved substrate solubility.

### Example 6

### Stability of the Catalase Immobilized Product

The immobilized catalase product was found to be heat resistant, as shown in FIG. 4. Heat treatment of 65° C. for 10 minutes destroyed the activity of free catalase while the immobilized catalase retained significant enzyme activity. However, extended incubations of the immobilized catalase product, such as incubations greater than 15 minutes, resulted in complete loss of catalase activity of the immobilized enzyme.

### Example 7

### Flow-through System Utilizing the Butyrylcholinesterase Immobilized Product

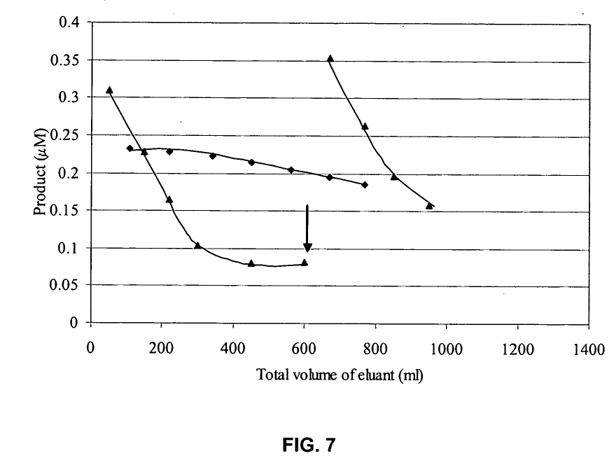

A stable immobilized enzyme system provides an opportunity to exploit a number of applications that would not be feasible with a soluble enzyme system. The stability of the immobilized butyrylcholinesterase product was demonstrated in a flow-through mini-reactor system in which the butyrylcholinesterase was continually reused over an extended period of time. The immobilized butyrylcholinesterase product was prepared in two alternative systems: a fluidized bed system and a packed-bed system. A flow rate of 1 ml·min⁻¹ was used in both the fluidized-bed and packed-bed systems. In both of these systems, complete conversion of the indophenyl acetate solution continued for over 1000 column volumes (500 ml) of the indophenyl acetate solution with no significant loss in enzyme activity or conversion efficiency, as shown in FIG. 7.

In the fluidized-bed system at 2 ml·min⁻, the reaction rate was sustained for over 2000 column volumes of the indophenyl acetate solution, again with little loss in activity. However, the conversion efficiency was lower (approximately 50%) due to the lower retention time within the column. In the packed-bed system, however, the conversion rate decreased with time, as shown in FIG. 7. The loss of activity could not be attributed to washout of the enzyme because no enzyme was detected in the eluate. When the packing material was removed, washed, resuspended, and returned to the column, the conversion rate returned to its original maximum and the activity decreased again during the second run demonstrating that the immobilized enzyme was still present and active. It was observed that the silica matrix from the packed-bed columns formed a solid plug that showed initial signs of cracking. It was concluded that the enzyme activity had not been reduced during the continuous flow, but rather the overall retention time was significantly lowered due to packing and eventual channeling of the silica particles.

The mechanical stability of the immobilized butyrylcholinesterase product indicated that it may be used in flow-through applications. However, the configuration of the apparatus remains to be optimized.

### Example 8

### Continuous Flow System Utilizing the Butyrylcholinesterase Immobilized Product

For continuous flow experiments, 0.5 ml polyethylene solid phase extraction tubes (available from Supelco) were packed with the butyrylcholinesterase immobilized product obtained from a 1 ml reaction mixture containing enzyme at a final concentration of 50 U/ml. The butyrylcholinesterase immobilized product was supported by 20 μm polyethylene frits (available from Supelco). A packed-bed reactor system and a fluidized-bed reactor system were investigated. The packed column was prepared by enclosing the butyrylcholinesterase immobilized product tightly between the frits. The packed column was washed with 5 ml of cholinesterase buffer before use. The indophenyl acetate solution (2×10⁻⁴ M final concentration in cholinesterase buffer, pH 8) was pumped through a 0.22 μM filter and then through the packed column at a fixed rate. The effluent was collected in 10 ml fractions in a fraction collector prior to determination of the absorbance of the blue reaction product.

The movement of the biosilica nanospheres in the fluidized bed column was created by the motion of liquid flowing upwards through the packed column; the volume in the fluidized-bed reactor was 0.5 ml. The quantity of biosilica-immobilized enzyme was the same in both cases. The entire apparatus was enclosed within an incubator and maintained at 25° C.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A method of immobilizing at least one molecule in a silica matrix, comprising:
combining at least one silaffin polypeptide, at least one molecule, and at least one hydroxylated water-soluble derivative to form a biosilicification product comprising the at least one molecule immobilized in the silica matrix.

2. The method of claim 1, wherein combining the at least one silaffin polypeptide, the at least one molecule, and the at least one hydroxylated water-soluble derivative comprises combining silicic acid with the at least one silaffin polypeptide and the at least one molecule.

3. The method of claim 1, wherein combining the at least one silaffin polypeptide, the at least one molecule, and the at least one hydroxylated water-soluble derivative comprises combining at least one silaffin polypeptide selected from the group consisting of Sil1 protein, a fragment of the Sil1 protein, a synthetic polypeptide capable of precipitating silica, and poly-L-lysine with the at least one molecule and the at least one hydroxylated water-soluble derivative.

4. The method of claim 1, wherein combining the at least one silaffin polypeptide, the at least one molecule, and the at least one hydroxylated water-soluble derivative comprises combining at least one silaffin polypeptide selected from the group consisting of a polypeptide having an amino acid sequence of SEQ ID NO:1, SEQ ID NO:2, SEQ ID NO:3, SEQ ID NO:4, SEQ ID NO:5, SEQ ID NO:6, and SEQ ID NO:7 with the at least one molecule and the at least one hydroxylated water-soluble derivative.

5. The method of claim 1, wherein combining the at least one silaffin polypeptide, the at least one molecule, and the at least one hydroxylated water-soluble derivative comprises combining a silaffin polypeptide having an amino acid sequence of SEQ ID NO:1 with the at least one molecule and the at least one hydroxylated water-soluble derivative.

6. The method of claim 1, wherein combining the at least one silaffin polypeptide, the at least one molecule, and the at least one hydroxylated water-soluble derivative comprises combining at least one organic molecule or at least one inorganic molecule with the at least one silaffin polypeptide and the at least one hydroxylated water-soluble derivative.

7. The method of claim 1, wherein combining the at least one silaffin polypeptide, the at least one molecule, and the at least one hydroxylated water-soluble derivative comprises combining at least one molecule selected from the group consisting of an enzyme, a protein, a polypeptide, an antibody, an antigen, poly(nucleic) acids, microbial cells, plant cells, and animal cells with the at least one silaffin polypeptide and the at least one hydroxylated water-soluble derivative.

8. The method of claim 1, wherein combining the at least one silaffin polypeptide, the at least one molecule, and the at least one hydroxylated water-soluble derivative comprises combining an enzyme selected from the group consisting of a hydrolase, a catalase, and an esterase with the at least one silaffin polypeptide and the at least one hydroxylated water-soluble derivative.

9. The method of claim 1, wherein combining the at least one silaffin polypeptide, the at least one molecule, and the at least one hydroxylated water-soluble derivative comprises combining an enzyme selected from the group consisting of butyrylcholinesterase, horseradish peroxidase, and catalase with the at least one silaffin polypeptide and the at least one hydroxylated water-soluble derivative.

10. The method of claim 1, wherein combining the at least one silaffin polypeptide, the at least one molecule, and the at least one hydroxylated water-soluble derivative comprises combining a magnetic material or a semiconductor material with the at least one silaffin polypeptide and the at least one hydroxylated water-soluble derivative.

11. The method of claim 1, wherein combining the at least one silaffin polypeptide, the at least one molecule, and the at least one hydroxylated water-soluble derivative comprises combining an inorganic material selected from the group consisting of cobalt platinum, iron oxide, titanium dioxide, cadmium selenide, cadmium sulfide, cadmium telluride, zinc selenide, zinc sulfide, and zinc telluride with the at least one silaffin polypeptide and the at least one hydroxylated water-soluble derivative.

12. The method of claim 1, wherein combining the at least one silaffin polypeptide, the at least one molecule, and the at least one hydroxylated water-soluble derivative comprises incorporating the at least one molecule into the silica matrix as the biosilicification product is formed.

13. The method of claim 1, wherein combining the at least one silaffin polypeptide, the at least one molecule, and the at least one hydroxylated water-soluble derivative comprises forming the silica matrix and immobilizing the at least one molecule within the silica matrix substantially simultaneously.

14. The method of claim 1, wherein combining the at least one silaffin polypeptide, the at least one molecule, and the at least one hydroxylated water-soluble derivative comprises forming the biosilicification product in which the at least one molecule is immobilized in the silica matrix.

15. The method of claim 1, wherein combining the at least one silaffin polypeptide, the at least one molecule, and the at least one hydroxylated water-soluble derivative comprises combining the at least one silaffin polypeptide, the at least one molecule, and the at least one hydroxylated water-soluble derivative at a pH ranging from approximately 5 to approximately 8.

16. The method of claim 1, wherein combining the at least one silaffin polypeptide, the at least one molecule, and the at least one hydroxylated water-soluble derivative comprises combining the at least one silaffin polypeptide, the at least one molecule, and the at least one hydroxylated water-soluble derivative at a pH of approximately 7.

17. The method of claim 1, wherein combining the at least one silaffin polypeptide, the at least one molecule, and the at least one hydroxylated water-soluble derivative comprises combining the at least one silaffin polypeptide, the at least one molecule, and the at least one hydroxylated water-soluble derivative at a temperature ranging from approximately 10° C. to approximately 25° C.

18. The method of claim 1, wherein combining the at least one silaffin polypeptide, the at least one molecule, and the at least one hydroxylated water-soluble derivative comprises combining the at least one silaffin polypeptide, the at least one molecule, and the at least one hydroxylated water-soluble derivative at ambient pressure.


SEQUENCE LISTING (7 sequences)

SEQ ID NO: 1 - protein, 19 aa, Cylindrotheca fusiformis
SSKKSGSYSGSKGSKRRIL

SEQ ID NO: 2 - protein, 12 aa, Artificial
  Peptide display library
APPGHHHWHIHH

SEQ ID NO: 3 - protein, 12 aa, Artificial
  Peptide display library
KPSHHHHHTGAN

SEQ ID NO: 4 - protein, 12 aa, Artificial
  Peptide display library
MSPHPHPRHHHT

SEQ ID NO: 5 - protein, 12 aa, Artificial
  Peptide display library
MSPHHMHHSHGH

SEQ ID NO: 6 - protein, 12 aa, Artificial Sequence
  Peptide display library
LPHHHHLHTKLP

SEQ ID NO: 7 - protein, 12 aa, Artificial
  Peptide display library
APHHHHPHHLSR